(19) United States
(12) Patent Application Publication
(10) Pub. No.: US 2026/0115002 A1
(43) Pub. Date: Apr. 30, 2026

(54) TRI-TAPER HIP STEM

(71) Applicant: GLOBUS MEDICAL, INC., AUDUBON, PA (US)

(72) Inventors: John Loiacono, Collegeville, PA (US); David Stumpo, Trappe, PA (US); Carly McGonagle, Philadelphia, PA (US)

(21) Appl. No.: 18/929,681

(22) Filed: Oct. 29, 2024

(51) Int. Cl. A61F 2/36

(52) U.S. Cl. CPC A61F 2/3662; A61F 2002/365

(57) ABSTRACT

Provided are hip stems for hip arthroplasty, kits of implant systems, and methods of using said hip stems and kits. The hip stems comprise a stem body having a length formed by a proximal portion, a middle portion, and a distal portion, and a stem neck extending from the proximal portion of the stem body and terminating at a free end. The stem portion comprises a first taper along a length of the middle portion of the stem body causing a width of the stem body to decrease, a second taper along the length of the stem body causing a width of the stem body in the posterior-anterior direction to decrease, and a third taper along a base of the stem neck causing a width of the base in the posterior-anterior direction to increase.

### FIELD OF THE INVENTION

The present disclosure relates to hip arthroplasty and, more specifically, to hip stems used in hip arthroplasty procedures.

### BACKGROUND OF THE INVENTION

Hip arthroplasty, often called hip replacement, is a surgical procedure used to reconstruct and resurface a hip joint that has been damaged by disease or injury (e.g., arthritis or hip fracture). Total hip arthroplasty (THA) devices may replace both the acetabulum and the femoral head which comprise the hip joint, where the femur articulates relative to the acetabulum. To replace the hip joint, the hip arthroplasty may include a femoral implant (or “hip stem”) secured to the end of the femur and an acetabular implant secured to the acetabulum that forms a replacement articulating surface which interfaces with the femoral implant. The femoral implant is pivotably coupled to the acetabular implant, thereby reconstructing the hip joint.

During a total hip arthroplasty, a femoral neck osteotomy may be performed to remove the femoral head. The surgeon will then locate the femoral canal, and progressively broach the bone until the cortical walls of the femur have been contacted. After a trial reduction is conducted to ensure the correct femoral head and neck offset is chosen, the stem of the implant (“hip stem”) is inserted into the broached canal. In the case of a cementless implant, the femoral implant, after impaction, creates a press fit between the implant and the cortical walls of the femur.

In some cases, the femoral implant, or hip stem, may subside into the medullary canal. For example, implant subsidence can happen in cementless stems when the press fit of the stem is not tight enough. Subsidence in the case of hip stems can lead to a periprosthetic fracture, in addition to patient pain, joint dislocation, and revision surgeries.

### SUMMARY OF THE INVENTION

Provided herein are tri-taper hip stems designed for reconstruction of the proximal femur in a hip arthroplasty. The tri-taper hip stems described herein are designed with a unique taper design and/or a medial collar that help mitigate the risk of subsidence otherwise associated with femoral implants. Mitigating risk of subsidence correspondingly reduces the risk of a periprosthetic fracture, pain, joint dislocation, and need for revision surgery often associated with implant subsidence as described above. More specifically, the hip stems provided are designed to form a tighter press fit formed by the implant's contact with the cortical bone in not only the medial and lateral aspects, but in also the anterior and posterior aspects. A tighter fit formed by an increased metaphyseal fill will minimize the likelihood of subsidence, which will in turn also reduce the chances of the negative affects associated with subsidence.

The tri-taper hip stems disclosed herein taper along three separate planes to more securely engage the proximal press fit of the cortical bone. Additionally, the tri-taper design of the hip stems described herein allows the stem to fit into all Dorr types of femurs, assuming the correct size of hip stem is used. In some embodiments, the tri-taper hip stems disclosed herein may also include a medial collar. The medial collar can further help decrease the likelihood of subsidence.

In some embodiments, the tri-taper hip stems may feature one or more surface coatings or surface treatments. For example, in some embodiments, a portion of the hip stem may be coated with a titanium plasma spray. A titanium plasma spray may help improve both short- and long-term fixation. In some embodiments, the tri-taper hip stem may have a hydroxyapatite (HA) coating. An HA coating can help promote bone growth. The tri-taper hip stem may be entirely coated with a HA coating or only partially coated with an HA coating. In some embodiments, a tri-taper hip stem may comprise both a titanium plasma spray coating and an HA coating. In some embodiments, a tri-taper hip stem may comprise only one of a titanium plasma spray coating or an HA coating.

Disclosed herein are both cementless and cemented hip stems. A cementless hip stem is one that may be implanted without cement and fixed to the bone through bone ingrowth. A cemented hip stem is secured to the bone with cement. Both types of hip stems are described herein, as well as the subtle design differences between them.

Provided is a hip stem for a hip arthroplasty, the hip stem comprising: a stem body having a length formed by a proximal portion, a middle portion, and a distal portion; and a stem neck extending from the proximal portion of the stem body and terminating at a free end, wherein the stem body comprises a first taper along a length of the stem body as measured in the cranial-caudal direction causing a width of the stem body in the medial-lateral direction to decrease as the first taper extends along the middle portion of the stem body, a second taper along the length of the stem body as measured in the cranial-caudal direction causing a width of the stem body in the posterior-anterior direction to decrease as the second taper extends along the middle portion of the stem body, and a third taper along a base of the stem neck causing a width of the base in the posterior-anterior direction to increase as the third taper extends along the base from the free end of the stem neck towards the stem body.

In some embodiments of the hip stem, the distal portion of the stem body comprises a taper in the medial-lateral direction.

In some embodiments of the hip stem, the first taper extends only through the middle portion.

In some embodiments of the hip stem, the first taper reduces a width of the middle portion of the hip stem in the medial-lateral direction by 30-50%.

In some embodiments of the hip stem, the second taper extends only through the proximal portion and the middle portion.

In some embodiments of the hip stem, the second taper reduces a width of the hip stem in the posterior-anterior direction by 20-50%.

In some embodiments of the hip stem, the third taper extends only through the proximal portion.

In some embodiments of the hip stem, the third taper enlarges a width of the hip stem in the posterior-anterior direction by 10-40%.

In some embodiments of the hip stem, the hip stem comprises a medial collar between the stem neck and the stem body extending medially downward.

In some embodiments of the hip stem, the medial collar is configured to sit along a calcar plane when implanted in a femur.

In some embodiments of the hip stem, the hip stem does not comprise a medial collar between the stem neck and the stem body.

In some embodiments of the hip stem, the hip stem comprises a drive pin hole in the base of the stem neck and extending into the proximal portion of the stem body.

In some embodiments of the hip stem, the drive pin hole comprises a key slot configured to be used for version control.

In some embodiments of the hip stem, the hip stem comprises a titanium plasma spray coating on a portion of the stem body.

In some embodiments of the hip stem, the hip stem comprises a hydroxyapatite coating on the entire stem body.

In some embodiments of the hip stem, the hip stem is a cementless, press-fit hip stem comprising a titanium alloy material.

In some embodiments of the hip stem, the hip stem is a cemented hip stem comprising a cobalt chrome material.

In some embodiments of the hip stem, a lateral surface of the middle portion of the stem body is at least twice as long as a lateral surface of the proximal portion of the stem body and a lateral surface of the distal portion of the stem body.

In some embodiments, provided is a kit for performing a hip arthroplasty, the kit comprising: a broaching system for preparing a femur for a total hip arthroplasty; a cementless hip stem; and a cemented hip stem, wherein both the cementless hip stem and the cemented hip stem comprise: a stem body having a length formed by a proximal portion, a middle portion, and a distal portion; and a stem neck extending from the proximal portion of the stem body and terminating at a free end, wherein the stem portion comprises a first taper along the length of the stem body causing a width of the stem body in the medial-lateral direction to decrease as the first taper extends along the middle portion of the stem body from a proximal end of the middle portion to a distal end of the middle portion, a second taper along the length of the stem body causing a width of the stem body in the posterior-anterior direction to decrease as the second taper extends along the middle portion of the stem body from the proximal end of the middle portion to the distal end of the middle portion, and a third taper along a base of the stem neck causing a width of the base in the posterior-anterior direction to increase as the third taper extends along the base from a proximal end of the stem neck towards the stem body, and wherein the broaching system can be used for both the cemented hip stem and the cementless hip stem.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1A shows a side view of a tri-taper hip stem, according to some embodiments;

FIG. 1B shows a front view of a tri-taper hip stem, according to some embodiments;

FIG. 1C shows a top view of a tri-taper hip stem, according to some embodiments;

FIG. 2 shows an outline overlay of a cemented tri-taper hip stem and a cementless tri-taper hip stem, according to some embodiments; and

FIG. 3 shows a side view of a tri-taper hip stem defining various dimensions, according to some embodiments.

### DETAILED DESCRIPTION OF THE DISCLOSURE

Provided herein are both cementless and cemented tri-taper hips stems for use in a hip arthroplasty. The hip stems described herein are designed to minimize the chances of subsidence which can occur when the bone near an implanted device settles or collapses and can cause a periprosthetic fracture, patient pain, joint dislocation, and need for revision surgery. In the case of subsidence of a hip stem, subsidence can occur when the fit of the hip stem is not tight enough, particularly in the case of a press fit or cementless stem.

Accordingly, the hip stems described herein have a taper design and, in some embodiments, a medial collar, both of which are designed to help reduce instances of subsidence. Each of these features are designed in more detail below.

First, the tri-taper hip stems described herein help prevent subsidence with a tri-taper design by filling the patient's femoral canal along more surfaces than other hip stems. Filling the femoral canal by providing more surface area contact between the hip stem and the femoral canal can provide a tighter fit, or a tighter press fit, of the hip stem into the patient's femur. The tightness of the fit of the hip stem in the patient's femoral canal is directly related to the chances of subsidence. Therefore, a tighter fight formed by providing more surface area contact between the surfaces of the hip stem and the bone of the femoral canal will decrease the likelihood of subsidence.

The higher surface area contact between the surfaces of the hip stem and the femoral canal is due to the taper design of the hip stem (“tri-taper”). These three different tapers can provide a tighter press fit due to the contact between the hip stem and the cortical bone in not only the medial and lateral aspect, which is typical in other hip stems, but also in the anterior and posterior aspects. The additional surface area contact particularly in the anterior and posterior aspects can help form a tighter fit or a tighter press fit of the hip stem in the femoral canal, minimizing the risk of subsidence.

Second, the tri-taper hip stems described herein can also help prevent subsidence by transferring the forces driving subsidence to the calcar of the femur, allowing the hip stem to stay in place and reduce the amount of subsidence. To transfer these forces, some embodiments of the hip stems described herein may include a medial collar. This medial collar is located at a proximal end of the hip stem between the body of the hip stem and a neck of the hip stem. The medial collar is optional.

During a total hip arthroplasty (THA), a femoral neck osteotomy may be performed to remove the femoral head after the correct size stem has been estimated, for example, in pre-operative templating. Following this, a reamer may be used to locate the femoral canal and broaching occurs until the cortical walls of the femur have been contacted. The broach sizes correlate to the size stems available. The surgeon utilizes the appropriate broach to choose the stem size more accurately. After a trial reduction is conducted to ensure the correct femoral head and neck offset is chosen, the stem is inserted into the broached canal. Trial reduction occurs with the broach left in the metaphysis. This may be used to assess leg length, range of motion, and joint stability. A cementless implant, after impaction, creates a press fit against the implant and the cortical walls of the femur. The stems have numerous sizes which may vary in the medial/lateral, anterior/posterior, and/or proximal/distal aspects. These size variations allow for a wide range of patients to receive the size stem most appropriate to their anatomy.

More recently, surgeons have been conducting total hip arthroplasties using a direct anterior approach. This approach can introduce a difficult angle for surgeons to insert the hip stem into the medullary canal. In some embodiments, a tri-taper hip stem according to some embodiments described herein may be designed with a relatively shorter stem with material relief on the lateral side of the distal tip. This geometry may allow the stem to be more easily inserted at the angle necessary when using a direct anterior approach.

Another benefit of the tri-taper hip stems described herein is that both the cementless tri-taper hip stems and the cemented tri-taper hip stems utilize the same broach system. This reduces the amount of instrumentation needed for the procedure and streamlines the entire process. It can also save operating room time, and in some cases, even preserves a patient's bone. In some embodiments, the cemented and cementless hip stems described herein may also utilize the same inserter(s), trial(s), and/or femoral head(s), further improving efficiency and decreasing the amount of instrumentation associated with the implant(s).

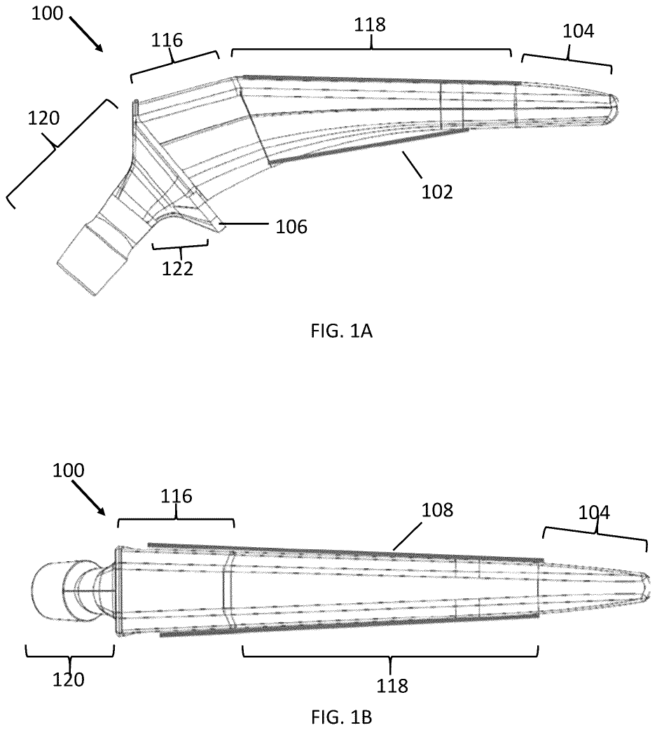

FIGS. 1A-1C show a side view, a front view, and a top view of a tri-taper hip stem 100 according to some embodiments. Each view is described in detail below.

FIG. 1A shows a side view of a tri-taper hip stem 100, according to some embodiments. Shown in this figure is a first taper 102, a distal end 104, and a medial collar 106. Also shown are proximal portion 116, middle portion 118, neck portion 120, and base 122 of neck portion 120.

As shown, hip stem 100 extends from neck portion 120 to distal end 104. Base 122 of neck portion 120 is located on a proximal portion of the neck portion 120. The proximal portion 116, middle portion 118, and distal portion 104 form the stem body. The proximal portion 116 is located immediately distal to the base 122 of neck portion 120 or medial collar 106, depending on whether the medial collar 106 is included. The middle portion 118 is immediately distal to proximal portion 116, and distal end 104 (or distal portion 104) is immediately distal to middle section 118.

In some embodiments, the length of middle portion 118 is greater than a length of each of proximal portion 116 and distal portion 104. In some embodiments, the length of middle portion 118 is at least double the length of proximal portion 116. In some embodiments, the length of middle portion 118 is at least double the length of distal portion 104. As used herein, the “length” of an individual portion of the stem body (e.g., proximal portion 116, middle portion 118, or distal portion 104) is measured along an axis extending through the center of the portion, generally (but not necessarily strictly) from the proximal end to the distal end (or in the cranial-caudal direction). By this definition, the axis of each of the proximal portion 116, the middle portion 118, and the distal portion 104 as shown in FIG. 1A runs along a different plane and no two axes of the three axes are co-axial.

In some embodiments, the lateral surface or edge of the proximal portion 116 runs along a first plane and the lateral surface or edge of middle portion 118 runs along a second plane, wherein the first plane and the second plane are not parallel and are not perpendicular. In some embodiments, the lateral surface or edge of the proximal portion 116 runs along a first plane and the lateral surface or edge of the distal portion 104 runs along a third plane, wherein the first plane and the third plane are not parallel and are not perpendicular. In some embodiments, the second plane and the third plane are not parallel and are not perpendicular. In some embodiments, the medial edge or surface of the proximal portion 116 runs along a fourth plane and the medial edge or surface of the middle portion 118 runs along a fifth plane, wherein the fourth plane and the fifth plane are not parallel and are not perpendicular. In some embodiments, the medial edge or surface of the proximal portion 116 runs along the fourth plane and the medial edge or surface of the distal portion 104 runs along a sixth plane, wherein the fourth plane is not parallel and is not perpendicular to the sixth plane. In some embodiments, the fifth plane and the sixth plane are not parallel and are not perpendicular. In some embodiments, no two planes of the six total planes (the first plane, the second plane, the third plane, the fourth plane, the fifth plane, and the sixth plane) are parallel. In some embodiments, no two planes of the six total planes (the first plane, the second plane, the third plane, the fourth plane, the fifth plane, and the sixth plane) are perpendicular.

The tri-taper design is described below with respect to the first taper 102, the second taper 108, and the third taper 110. Each taper “extends” insofar as each of the involved surfaces of the hip stem 100 extend planarly. (However, by definition, the first surface of a taper will extend along a different plane than the second, opposing surface of said taper.) In one example, the first taper 102 as shown in FIG. 1A extends only along the middle portion 118. As is shown in the figure, and particularly on the lateral side of the hip stem 100, the lateral surface of proximal portion 116 and the lateral surface of distal portion 104 extend along different planes than the plane of the first taper 102 of the lateral surface of middle portion 118 of hip stem 100. However, as described in further detail below, this is only one embodiment of hip stem 100 and first taper 102.

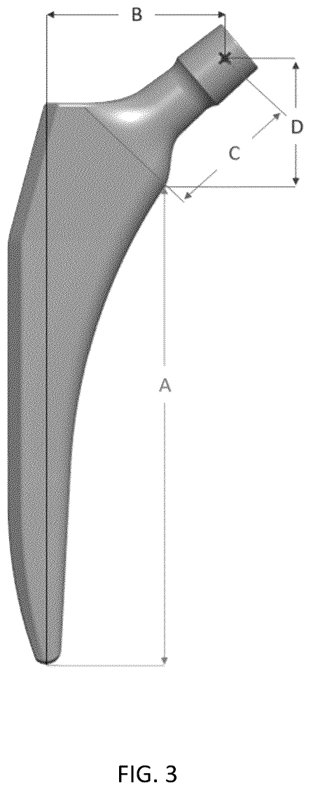

The first taper 102 extends along a length of the hip stem 100 such that a width of the hip stem in the medial-lateral direction narrows as the first taper 102 extends towards the distal portion of the hip stem 100. As used herein the “length of the hip stem” is measured in the direction labeled “A” in FIG. 3. (Note that this is different than a “length” of an individual portion of the stem body.) In some embodiments, the first taper 102 extends through the proximal portion of the hip stem 100. In some embodiments, the first taper extends only through the proximal portion of the hip stem 100. In some embodiments, the taper angle of the first taper 102 is the same along both the medial edge and lateral edge of the hip stem 100. In some embodiments, the taper angle of the first taper 102 is different along each of the medial edge and the lateral edge of the hip stem 100. In some embodiments, the taper angle of the first taper 102 along the medial edge of the hip stem 100 is greater than the taper angle of the first taper 102 along the lateral edge of the hip stem 100. In some embodiments, the medial edge of the hip stem 100 is curved.

In some embodiments, the first taper 102 reduces the width of the hip stem in the medial-lateral direction by 20-60% or by 30-50%. In some embodiments, the first taper 102 reduces the width of the hip stem in the medial-lateral direction by less than or equal to 60%, less than or equal to 55%, less than or equal to 50%, less than or equal to 45%, less than or equal to 40%, less than or equal to 35%, less than or equal to 30%, or less than or equal to 25%. In some embodiments, the first taper 102 reduces the width of the hip stem in the medial-lateral direction by greater than or equal to 20%, greater than or equal to 25%, greater than or equal to 30%, greater than or equal to 35%, greater than or equal to 40%, greater than or equal to 45%, greater than or equal to 50%, or greater than or equal to 55%.

The distal end 104 of the hip stem features a taper in the medial-lateral aspect to relieve material for easy insertion of the stem. This relief cut may be featured on the lateral side of the hip stem 100. As explained above, a direct anterior approach, which is being use more frequently, introduces a challenging angle for hip stem insertion due to patient anatomy. However, a taper in the medial-lateral aspect on the distal end 104 of the hip stem 100 can help a surgeon more easily insert the hip stem into the medullary canal of a patient's femur.

Some embodiments of a tri-taper hip stem 100 can include a medial collar 106. The medial collar 106 is optional, and it is designed to distribute axial forces over the calcar plane in order to minimize the possibility of implant subsidence. More specifically, the medial collar 106 is designed to sit on the calcar of the patient's femur when properly implanted. If the hip stem begins to subside, forces will be transferred to the calcar and the hip stem will stay in place.

FIG. 1B shows a front view of a tri-taper hip stem 100, according to some embodiments. FIG. 1B specifically shows the second taper 108 of the tri-taper design, as well as the neck portion 120, proximal portion 116, middle portion 118, and distal portion 104.

The second taper 108 extends along the length of the hip stem 100 such that a width of the hip stem in the posterior-anterior direction narrows as the second taper 108 extends towards the distal portion of the hip stem 100. As used herein the “length of the hip stem” is measured in the direction labeled “A” in FIG. 3. (Note that this is different than a “length” of an individual portion of the stem body.) In some embodiments, the second taper 108 extends along the proximal portion, the middle portion, and the distal portion of the hip stem 100. In some embodiments, the second taper 108 extends only along the proximal portion and the middle portion of the hip stem 100. In some embodiments, the taper angle along the anterior surface of the hip stem 100 is the same as the taper angle along the posterior surface of the hip stem 100. In some embodiments, the taper angle along the anterior surface of the hip stem 100 is different than the taper angle along the posterior surface of the hip stem 100.

In some embodiments, the second taper 108 reduces the width of the hip stem 100 in the posterior-anterior direction by 20-50%. In some embodiments, the second taper 108 reduces the width of the hip stem 100 in the posterior-anterior direction by less than or equal to 50%, less than or equal to 45%, less than or equal to 40%, less than or equal to 35%, less than or equal to 30%, or less than or equal to 25%. In some embodiments, the second taper 108 reduces the width of the hip stem 100 in the posterior-anterior direction by greater than or equal to 20%, greater than or equal to 25%, greater than or equal to 30%, greater than or equal to 35%, greater than or equal to 40%, or greater than or equal to 45%.

FIG. 1C shows a top view of a tri-taper hip stem 100, according to some embodiments. Specifically, FIG. 1C shows the third taper 110 and a key slot inserter hole 112.

The third taper 110 of the tri-taper design is designed to engage the press fit in the anterior and posterior cortical bone. Many hip stems are only configured to press fit in the medial and lateral directions. However, the third taper allows the tri-taper hip stem 100 to achieve a press fit in not only the medial and lateral aspects, but also in the anterior and posterior aspects. Achieving a press fit in the medial and lateral direction as well as in the anterior and posterior directions allow for a tighter fit of the hip stem and a reduced chance of subsidence.

In some embodiments, the third taper 110 of hip stem 100 extends along an axis of the proximal portion of hip stem 100 such that a width of the hip stem in the medial-lateral direction widens as the third taper 110 extends from the stem neck 120 towards the middle portion 118 of the hip stem 100. In some embodiments, the third taper 110 extends through the proximal portion of the hip stem 100. In some embodiments, the third taper 110 extends only through the proximal portion 116 of the hip stem 100. In some embodiments, the taper angle of the third taper 110 is the same along both the posterior surface and the anterior surface of the proximal portion 116 of hip stem 100. In some embodiments, the taper angle of the third taper 110 is the same along each of the posterior surface and the anterior surface of the hip stem 100. In some embodiments, the taper angle of the third taper 110 along the posterior surface of the hip stem 100 is different than the taper angle of the third taper 110 along the anterior surface of the hip stem 100.

In some embodiments, the third taper 110 enlarges the width of the hip stem 100 in the posterior-anterior direction by 20-80% or 10-40%. In some embodiments, the third taper 110 enlarges the width of the hip stem 100 in the posterior-anterior direction by less than or equal to 80%, less than or equal to 70%, less than or equal to 60%, less than or equal to 50%, less than or equal to 40%, less than or equal to 30%, or less than or equal to 20%. In some embodiments, the third taper 110 enlarges the width of the hip stem 100 in the posterior-anterior direction by greater than or equal to 10%, greater than or equal to 20%, greater than or equal to 30%, greater than or equal to 40%, greater than or equal to 50%, greater than or equal to 60%, or greater than or equal to 70%.

In some embodiments, a tri-taper hip stem 100 may include a key slot inserter hole 110. The key slot inserter hole 112 can allow for version control of the implant during insertion. Version control refers to the position of the hip stem 100 in the femoral canal. If the hip stem 100 is not placed correctly, it will be anteverted or retroverted. The key slot inserter hole 112 can help a surgeon maintain proper alignment during insertion and reduce the chances of an anteverted or retroverted implant. In some embodiments, a key slot can be machined in the medial-lateral aspect in addition to the proximal threaded drive pin hole.

Further, some embodiments of the hip stems described herein may include one or more surface coatings or surface treatments. Surface coatings can include, for example, a titanium plasma spray and/or a hydroxyapatite coating. In some embodiments, a portion of a hip stem might be coated with a titanium plasma spray coating. The portion of the hip stem coated with a titanium plasma spray may comprise the stem body. More specifically, it may comprise a proximal portion (e.g., proximal portion 116) of the stem body of the hip stem. In some embodiments, the titanium plasma spray may also coat a portion of or the entire surface of the middle portion (e.g., middle portion 118) of the hip stem. In some embodiments, a hip stem may be entirely coated with a hydroxyapatite coating or only partially coated with a hydroxyapatite coating. In some embodiments, a stem body of a hip stem may be entirely coated with a hydroxyapatite coating. In some embodiments, a stem neck (including a base of the stem neck) may not be coated with a hydroxyapatite coating. In some embodiments, only cementless hip stems may include one or more surface coatings. In some embodiments, both cementless and cemented hip stems may include one or more coatings. The titanium plasma spray coating can improve fixation (both short-term and long-term fixation). The hydroxyapatite coating can promote bone regrowth. In some embodiments, a tri-taper hip stem may comprise both a titanium plasma spray coating and an HA coating. In some embodiments, a tri-taper hip stem may comprise only one of a titanium plasma spray coating or an HA coating.

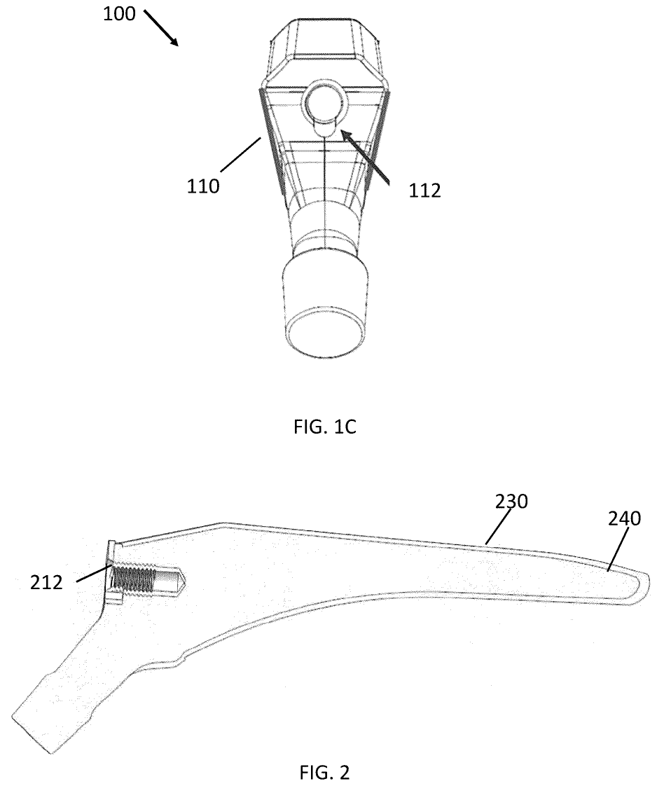

FIG. 2 shows an overlay of a cementless tri-taper hip stem 230 and a cemented tri-taper hip stem 240 of the same size. As used herein, “same size” means that a cementless hip stem 230 that is the “same size” as a cemented hip stem 240 may be used interchangeably in a patient with a specific anatomy. However, as shown in FIG. 2, the cemented hip stem 240 is slightly smaller than the cementless hip stem 230 to allow space for the cement to fit between the patient's bone and the implant. In some embodiments, the cemented hip stem 240 may be 2 mm smaller around all surfaces of the stem body to allow for the cement mantle. However, the neck 120 and base 122 may be the same dimensions between the cementless hip stem 230 and the cemented hip stem 240 such that all they are both compatible with existing femoral heads (e.g., Globus Medical's existing femoral heads). Having the cemented option that uses the same broaches as the press fit stem will reduce the amount of instrumentation that hospitals and surgery centers need to have. The hip stems described herein (both cementless and cemented) may also utilize the same inserter(s), trial neck(s), and/or femoral head(s), further reducing the necessary amount of instrumentation. It will also save surgeons time in the operating room when they decide to switch to a cemented stem since they won't have to re-broach or ream and will preserve the patient's bone.

The hip stems described herein may comprise one or more biocompatible materials. For example, a hip stem according to some embodiments may be made from a metal (e.g., titanium, stainless steel, cobalt chrome, carbon composite, or suitable alloys), a plastic or polymer (e.g., polyethylene, ultra-high molecular weight polyethylene (UHMWPE), polyetheretherketone (PEEK)), or combinations of such materials. These parts may be machined, constructed from additive manufacturing, such as 3D printing, subtractive manufacturing, or hybrid manufacturing processes. Although the materials described herein are exemplified, it will be appreciated that any suitable materials and construction may be selected for the individual components.

### Kits

Also provided herein are kits for use in a hip arthroplasty. In some embodiments, a kit can include a broaching system and two or more hip stems. The broaching system may be configured for preparing a femur for a total hip arthroplasty. The two or more hip stems may include one or more of: two or more sizes of cementless hip stems, two or more sizes of cemented hip stems, or a cementless hip stem and a cemented hip stem of the same size. In some embodiments, the kit comprises all of: two or more sizes of cementless hip stems, two or more sizes of cemented hip stems, and a cementless hip stem and a cemented hip stem of the same size. In some embodiments, all of the hip stems in the kit comprise a stem body having a length formed by a proximal portion, a middle portion, and a distal portion; and a stem neck extending from the proximal portion of the stem body and terminating at a free end, wherein the stem portion comprises a first taper along the length of the stem body causing a width of the stem body in the medial-lateral direction to decrease as the first taper extends along the middle portion of the stem body from a proximal end of the middle portion to a distal end of the middle portion, a second taper along the length of the stem body causing a width of the stem body in the posterior-anterior direction to decrease as the second taper extends along the middle portion of the stem body from the proximal end of the middle portion to the distal end of the middle portion, and a third taper along a base of the stem neck causing a width of the base in the posterior-anterior direction to increase as the third taper extends along the base from a proximal end of the stem neck towards the stem body, and wherein the broaching system can be used for both the cemented hip stem and the cementless hip stem.

The kits provided herein can also include other implants (e.g., acetabular shell, liner, femoral head), various instruments, and other components for performing the procedure.

### Methods

Provided herein are methods for using the hip stems described in detail above. In some embodiments, a method for a hip arthroplasty may include one or more of the following steps in any suitable order: (1) performing a femoral neck osteotomy to remove a femoral head of a femur; (2) inserting a reamer into a femoral canal and broaching the bone until cortical walls of the femur are contacted; (3) trialing a reduction to assess leg length and/or joint stability; and (4) impacting a cementless tri-taper hip stem into the femoral canal.

In some embodiments, a method for a hip arthroplasty may include one or more of the following steps in any suitable order: (1) performing a femoral neck osteotomy to remove a femoral head of a femur; (2) inserting a reamer into a femoral canal and broaching the bone until cortical walls of the femur are contacted; (3) trialing a reduction to assess leg length and/or joint stability; (4) filling the femoral canal with cement; and (5) inserting a cemented tri-taper hip stem into the femoral canal.

### EXAMPLES

### Example 1: Press Fit (Cementless) Tri-Taper Hip Stems

Table 1, below, shows specific dimensions for cemented tri-taper hip stems sizes 1-12. The dimensions A, B, C, and D refer to the measurements labeled in FIG. 3.

TABLE 1
Cemented Tri-Taper Hip Stems Sizes 1-12
Press Fit Stems

| | | B | | | | | D |
|---|---|---|---|---|---|---|---|
| | | Horizontal offset from | | | C | | Vertical |
| | A | Stem Centerline (Offset) | | | Neck Length | | Offset |
| Size | Length | STD | LAT | Offset | STD | LAT | (Leg Length) |
| 1 | 97 | 36.2 | 42.2 | 6 | 28 | 32 | 27 |
| 2 | 99 | 36.2 | 42.2 | 6 | 28 | 32 | 27 |
| 3 | 101 | 37.4 | 43.4 | 6 | 29 | 33.5 | 28 |
| 4 | 103 | 37.4 | 43.4 | 6 | 29 | 33.5 | 28 |
| 5 | 105 | 38.6 | 44.6 | 6 | 30.5 | 34.75 | 29 |
| 6 | 107 | 38.6 | 44.6 | 6 | 30.5 | 34.75 | 29 |
| 7 | 109 | 39.75 | 45.75 | 6 | 32 | 36 | 30 |
| 8 | 111 | 39.75 | 45.75 | 6 | 32 | 36 | 30 |
| 9 | 113 | 40.9 | 46.9 | 6 | 33 | 37.5 | 31 |
| 10 | 115 | 40.9 | 46.9 | 6 | 33 | 37.5 | 31 |
| 11 | 117 | 42.1 | 48.1 | 6 | 34.5 | 38.8 | 32 |
| 12 | 119 | 42.1 | 48.1 | 6 | 34.5 | 38.8 | 32 |

Table 2 below shows specific dimensions for cementless tri-taper hip stems sizes 1-12. The dimensions A, B, C, and D refer to the measurements labeled in FIG. 3. As seen in the table, the actual dimensions of a cemented hip stem of a given size are smaller than the corresponding cementless (press fit) hip stem of the same size.

TABLE 2
Cementless Tri-Taper Hip Stems Sizes 1-12
Cemented Stems

| | | B | | | | | D |
|---|---|---|---|---|---|---|---|
| | | Horizontal offset from | | | C | | Vertical |
| | A | Stem Centerline (Offset) | | | Neck Length | | Offset |
| Size | Length | STD | LAT | Offset | STD | LAT | (Leg Length) |
| 3 | 95 | 37.4 | 43.4 | 6 | 29 | 33.5 | 28 |
| 4 | 97 | 37.4 | 43.4 | 6 | 29 | 33.5 | 28 |
| 5 | 101 | 38.6 | 44.6 | 6 | 30.5 | 34.75 | 29 |
| 6 | 103 | 38.6 | 44.6 | 6 | 30.5 | 34.75 | 29 |
| 7 | 105 | 39.75 | 45.75 | 6 | 32 | 36 | 30 |
| 8 | 107 | 39.75 | 45.75 | 6 | 32 | 36 | 30 |
| 9 | 109 | 40.9 | 46.9 | 6 | 33 | 37.5 | 31 |
| 10 | 111 | 40.9 | 46.9 | 6 | 33 | 37.5 | 31 |
| 11 | 113 | 42.1 | 48.1 | 6 | 34.5 | 38.8 | 32 |
| 12 | 115 | 42.1 | 48.1 | 6 | 34.5 | 38.8 | 32 |

Additional aspects, advantages and/or other features of example embodiments of the invention will become apparent in view of this detailed description. It should be apparent to those skilled in the art that the described embodiments provided herein are merely exemplary and illustrative and not limiting. Numerous embodiments and modifications thereof are contemplated as falling within the scope of this disclosure and equivalents thereto.

Further, although the invention has been described in detail and with reference to specific embodiments, it will be apparent to one skilled in the art that various changes and modifications can be made without departing from the spirit and scope of the invention. Thus, it is intended that the invention covers the modifications and variations of this invention provided they come within the scope of the appended claims and their equivalents. It is expressly intended, for example, that all components of the various devices disclosed above may be combined or modified in any suitable configuration.

1. A hip stem for a hip arthroplasty, the hip stem comprising:
a stem body having a length formed by a proximal portion, a middle portion, and a distal portion; and
a stem neck extending from the proximal portion of the stem body and terminating at a free end,
wherein the stem body comprises a first taper along a length of the stem body as measured in the cranial-caudal direction causing a width of the stem body in the medial-lateral direction to decrease as the first taper extends along the middle portion of the stem body, a second taper along the length of the stem body as measured in the cranial-caudal direction causing a width of the stem body in the posterior-anterior direction to decrease as the second taper extends along the middle portion of the stem body, and a third taper along a base of the stem neck causing a width of the base in the posterior-anterior direction to increase as the third taper extends along the base from the free end of the stem neck towards the stem body.

2. The hip stem of claim 1, wherein the distal portion of the stem body comprises a taper in the medial-lateral direction.

3. The hip stem of claim 1, wherein the first taper extends only through the middle portion.

4. The hip stem of claim 3, wherein the first taper reduces a width of the middle portion of the hip stem in the medial-lateral direction by 30-50%.

5. The hip stem of claim 1, wherein the second taper extends only through the proximal portion and the middle portion.

6. The hip stem of claim 5, wherein the second taper reduces a width of the hip stem in the posterior-anterior direction by 20-50%.

7. The hip stem of claim 1, wherein the third taper extends only through the proximal portion.

8. The hip stem of claim 7, wherein the third taper enlarges a width of the hip stem in the posterior-anterior direction by 10-40%.

9. The hip stem of claim 1, comprising a medial collar between the stem neck and the stem body extending medially downward.

10. The hip stem of claim 9, wherein the medial collar is configured to sit along a calcar plane when implanted in a femur.

11. The hip stem of claim 1, wherein the hip step does not comprise a medial collar between the stem neck and the stem body.

12. The hip stem of claim 1, comprising a drive pin hole in the base of the stem neck and extending into the proximal portion of the stem body.

13. The hip stem of claim 12, wherein the drive pin hole comprises a key slot configured to be used for version control.

14. The hip stem of claim 1, comprising a titanium plasma spray coating on a portion of the stem body.

15. The hip stem of claim 1, comprising hydroxyapatite coating on the entire stem body.

16. The hip stem of claim 1, wherein the hip stem is a cementless, press-fit hip stem comprising a titanium alloy material.

17. The hip stem of claim 1, wherein the hip stem is a cemented hip stem comprising a cobalt chrome material.

18. The hip stem of claim 1, wherein a lateral surface of the middle portion of the stem body is at least twice as long as a lateral surface of the proximal portion of the stem body and a lateral surface of the distal portion of the stem body.

19. A kit for performing a hip arthroplasty, the kit comprising:
a broaching system for preparing a femur for a total hip arthroplasty;
a cementless hip stem; and
a cemented hip stem, wherein both the cementless hip stem and the cemented hip stem comprise:
a stem body having a length formed by a proximal portion, a middle portion, and a distal portion; and
a stem neck extending from the proximal portion of the stem body and terminating at a free end,
wherein the stem portion comprises a first taper along the length of the stem body causing a width of the stem body in the medial-lateral direction to decrease as the first taper extends along the middle portion of the stem body from a proximal end of the middle portion to a distal end of the middle portion, a second taper along the length of the stem body causing a width of the stem body in the posterior-anterior direction to decrease as the second taper extends along the middle portion of the stem body from the proximal end of the middle portion to the distal end of the middle portion, and a third taper along a base of the stem neck causing a width of the base in the posterior-anterior direction to increase as the third taper extends along the base from a proximal end of the stem neck towards the stem body, and wherein the broaching system can be used for both the cemented hip stem and the cementless hip stem.
